(19) United States
(12) Patent Application Publication
(10) Pub. No.: US 2025/0325793 A1
(43) Pub. Date: Oct. 23, 2025

(54) INTRACALVARIOSSEOUS DRUG ADMINISTRATION DEVICE FOR DELIVERING DRUG BY BYPASSING BLOOD-BRAIN BARRIER

(71) Applicant: GACHON UNIVERSITY OF INDUSTRY-ACADEMIC COOPERATION FOUNDATION, Seongnam-si (KR)

(72) Inventors: Young Tag KO, Gimpo-si (KR); Ji Hee KANG, Incheon (KR)

(21) Appl. No.: 19/253,922

(22) Filed: Jun. 29, 2025

Related U.S. Application Data
(63) Continuation of application No. PCT/KR2023/013636, filed on Sep. 12, 2023.

(30) Foreign Application Priority Data
Jan. 5, 2023 (KR) 10-2023-0001859
Apr. 4, 2023 (KR) 10-2023-0044147

(51) Int. Cl. A61M 39/02

(52) U.S. Cl. CPC A61M 39/0247; A61M 2039/0205; A61M 2039/025; A61M 2039/0261; A61M 2205/18; A61M 2205/3327; A61M 2205/3344; A61M 2210/0687

(57) ABSTRACT

Provided is an intracranial drug administration device for delivering drug to a brain by bypassing a blood-brain barrier, and particularly, to a device for administering a drug to a diploe within the skull, including a receiving portion having a space provided therein for receiving the drug, and an injection portion formed by extending from the receiving portion to a predetermined length and allowing the drug received in the receiving portion to be discharged, in which an end portion of the injection portion is located in the diploe within the skull in a state where the receiving portion is located outside the skull.

### TECHNICAL FIELD

The present disclosure relates to an intracranial drug administration device for delivering a drug to a brain by bypassing a blood-brain barrier.

### BACKGROUND ART

Central nervous system (CNS) disorders account for approximately 6.3% of the global disease burden.

Despite advances in understanding the brain, including the structure and function of the CNS, over the past several decades, the discovery and clinical development of new drugs to treat CNS disorders remain a major challenge in the field of neuropharmacology.

The probability of a drug being developed for a central nervous system disease reaching the market is approximately twice as low as that of other disease treatments, while the development and approval period is approximately twice as long.

Advances in the treatment of central nervous system disorders are delayed due to limited understanding of brain disease pathology, and the reasons for the low treatment success rate include minimal understanding of the relevant pathophysiology of central nervous system disorders or difficulties in developing preclinical models and assessing target engagement, the presence of the blood-brain barrier (BBB), and aspects of central nervous system mediation.

In particular, the presence of the BBB, which consists of endothelial cells connecting tight junctions, is a major obstacle to drug efficacy, lack of clinical measures of sensitivity, interference of the placebo effect with the therapeutic effect, and drug entry into the brain.

The BBB is a unique structure that regulates inflow and outflow to maintain homeostasis and protect the brain from pathogenic substances, thereby limiting the entry of molecules into the brain.

Therefore, when the central nervous system disorder treatment drugs are administered by the general intravenous (IV) administration method, the degree of drug delivery to the brain due to the BBB is significantly low.

In order to overcome the BBB of the central nervous system disorder treatment drugs, in KR 10-2270248 B1 and KR 10-2021-0090433 A, a device is disclosed that directly administers drugs into the intracerebroventricular (ICV) as a direct invasive administration method.

The intracerebroventricular drug administration using this device has the advantage of overcoming the BBB and directly delivering drugs to the brain, but it is very invasive and may cause problems such as complications because a tube for injecting the drug should be inserted into the intracerebroventricular.

In addition, there is an intrathecal (IT) administration method that is not intracerebroventricular as the direct invasive administration method, but this method also has the same problems as the intracerebroventricular administration described above.

### DISCLOSURE

### Technical Problem

The present disclosure is devised to solve the above-mentioned conventional problems, and the inventors of the present disclosure, based on research, have found that the skull is not completely impermeable to drug penetration into the brain, and have sought to provide a drug administration device for intracalvariosseous (ICO) administration of drugs as a new approach for brain drug delivery through a bypass route of the blood-brain barrier (BBB).

### Technical Solution

In order to achieve the above-described objects, the present disclosure provides a device for administering a drug to a diploe within the skull, the device including: a receiving portion having a space provided therein for receiving the drug; and an injection portion formed by extending from the receiving portion to a predetermined length and allowing the drug received in the receiving portion to be discharged, in which an end portion of the injection portion is located in the diploe within the skull in a state where the receiving portion is located outside the skull.

Preferably, the device further includes a fixture 200 located on a peripheral side of the receiving portion.

Moreover, the present disclosure provides a device for administering a drug to a diploe within a skull, the device including: a receiving portion having a space provided therein for receiving the drug; an injection portion formed by extending from the receiving portion to a predetermined length and allowing the drug received in the receiving portion to be discharged; a holder fixed to an outside of the skull; a insertion portion formed by extending from the holder to a predetermined length and having a diameter larger than that of the injection portion; and a coupling hole located on a peripheral side of the holder, in which the holder is fixed to the outside of the skull by a coupling member coupled to the coupling hole, and in a state where the insertion portion is located in the diploe within the skull, the injection portion is inserted into the insertion portion so that the receiving portion is fixed to the outside of the skull, and an end portion of the injection portion is located in the diploe within the skull.

Moreover, the present disclosure provides a device for administering a drug to a diploe within a skull, the device including: a main body; an injection portion extending from the main body to a predetermined length; an injection tube installed by penetrating an end portion of the injection portion from the upper portion of the main body; a holder fixed to an outside of the skull; a fixing portion extending from the holder to a predetermined length and formed with a diameter larger than the diameter of the injection portion; and a coupling hole located on a peripheral side of the holder, in which the holder is fixed to the outside of the skull by a coupling member coupled to the coupling hole, and in a state where the insertion portion is located in the diploe within the skull, the receiving portion is fixed to the outside of the skull, and the injection portion is inserted into the insertion portion so that an end portion of the injection tube is located in the diploe within the skull.

Preferably, the device further includes a coupling hole located on the peripheral side of the holder, in which the holder is fixed to the outside of the skull as a coupling member penetrates the coupling hole and is coupled to the skull.

Preferably, the device further include screw threads formed on an outer surface of the injection portion and an inner surface of the fixing portion, respectively, in which the injection portion and the insertion portion are rotatably coupled by the screw threads.

Preferably, the predetermined length of the injection portion is 0.8 to 6.2 mm.

Preferably, the predetermined length of the injection portion is 0.8 to 6.2 mm added to a thickness of the holder.

Preferably, the device further includes: a pressure sensor located on a side of an end portion of the injection portion; and a notification unit that notifies when a pressure measured by the pressure sensor is within a preset pressure range.

### Advantageous Effects

The drug administration device according to the present disclosure provides an innovative and advantageous route of the drug for the effective treatment of brain diseases by allowing the drug to be administered into the skull, thereby solving both the problems of invasive procedures and complications of existing intracerebroventricular or intrathecal drug administration and the low drug delivery rate of intravenous administration.

### DESCRIPTION OF DRAWINGS

FIG. 1 is a perspective view of a drug administration device according to a first embodiment of the present disclosure.

FIG. 2 is a longitudinal cross-sectional view illustrating the drug administration device according to the first embodiment of the present disclosure installed in a skull.

FIG. 3 is a perspective view of a drug administration device according to a second embodiment of the present disclosure.

FIG. 4 is an exploded perspective view of the drug administration device according to the second embodiment of the present disclosure.

FIG. 5 is a longitudinal cross-sectional view illustrating the drug administration device according to the second embodiment of the present disclosure installed in the skull.

FIG. 6 is a perspective view of a drug administration device according to a third embodiment of the present disclosure.

FIG. 7 is an exploded perspective view of the drug administration device according to the third embodiment of the present disclosure.

FIG. 8 is a longitudinal cross-sectional view illustrating the drug administration device according to the third embodiment of the present disclosure installed in the skull.

FIG. 9 is a schematic diagram of a dorsal skull surface of a rat illustrating a location of a thinned skull area for intracalvariosseous (ICO) administration and a guide cannula for inserting a microdialysis probe.

FIG. 10 is a cross-sectional schematic diagram of the skull surface of a rat illustrating a location of the drug administration device according to the present disclosure and the microdialysis probe inserted into the brain.

FIG. 11 is a view of the drug administration device according to the present disclosure installed in a rat, illustrating the process of thinning the skull, inserting the drug administration device into the thinned skull area, fixing the drug administration device with a fixture, and then performing skin suturing.

FIG. 12 is a near-infrared (NIR) camera image after diffusion of indocyanine green (ICG) only in the left parietal bone 24 hours after intracalvariosseous drug administration.

FIG. 13 is a table illustrating the pharmacokinetic parameters and in vivo brain availability (F_{br}) of nine types of drugs (CPZ, RIS, DPZ, RVG, TMZ, PTX, GABA, GSH, and SUC) after intravenous (IV) and intracalvariosseous (ICO) administration.

FIGS. 14 to 22 illustrate the total brain drug concentration (C_{br}) and AUC ratio at 24 hours after ICO and IV administration of the nine types of drugs (CPZ, RIS, DPZ, RVG, TMZ, PTX, GABA, GSH, and SUC), respectively.

FIGS. 23 and 24 illustrate relationships between experimental brain drug concentrations (C_{br}) and in vitro experimental BBB permeability product-surface (PS) values, molecular weight (MW), and octanol-water partition coefficient (log Pow) at 24 hours after IV and ICO administration.

FIGS. 25 and 26 illustrate the relationship between the brain/plasma concentration ratio (Kₚ) and the experimental BBB permeability product-surface (PS) value, molecular weight (MW), and octanol-water partition coefficient (log Pow) calculated at 24 hours after IV and ICO administration.

FIG. 27 illustrates the main transcranial pathways of molecules penetrating from the skull cortex to the brain after ICO administration.

### BEST MODE

The above-described purposes, features and other advantages of the present disclosure will become more apparent by describing the preferred embodiments of the present disclosure in detail with reference to the attached drawings. In this process, the thickness of the lines and the sizes of the components illustrated in the drawings may be exaggerated for the sake of clarity and convenience of explanation. In addition, the terms described below are terms defined in consideration of the functions of the present disclosure, and may vary depending on the intention or custom of the user or operator. Therefore, the definitions of these terms should be described based on the contents throughout this specification.

In addition, the described embodiments are provided as examples for the purpose of explaining the disclosure, and do not limit the technical scope of the present disclosure.

In the entire specification of the present disclosure, when a part is said to be “connected” to another part, this includes not only the case where it is “directly connected”, but also the case where it is “electrically connected” with another element in between.

Throughout the present specification, when a component is said to be located “on”, “above”, “upper end”, “below”, “below”, or “lower end” another component, this includes not only cases where a component is in contact with another component, but also cases where another component exists between the two components.

Throughout the present specification, when a part is said to “include” a component, this does not mean that other components are excluded, unless otherwise specifically stated, but rather that other components may be included.

In addition, terms related to direction or position (upper side, upper surface, lower side, or the like) in the description of the embodiments of the present disclosure are set based on the arrangement of each component illustrated in the drawings.

Hereinafter, a drug administration device according to a preferred embodiment of the present disclosure will be described in detail with reference to the attached drawings.

The drug administration device according to the present disclosure is a device for administering a drug to a diploe of the skull. The diploe is a layer containing bone marrow located between an outer cortex and an inner cortex of the skull.

### 1. First Embodiment

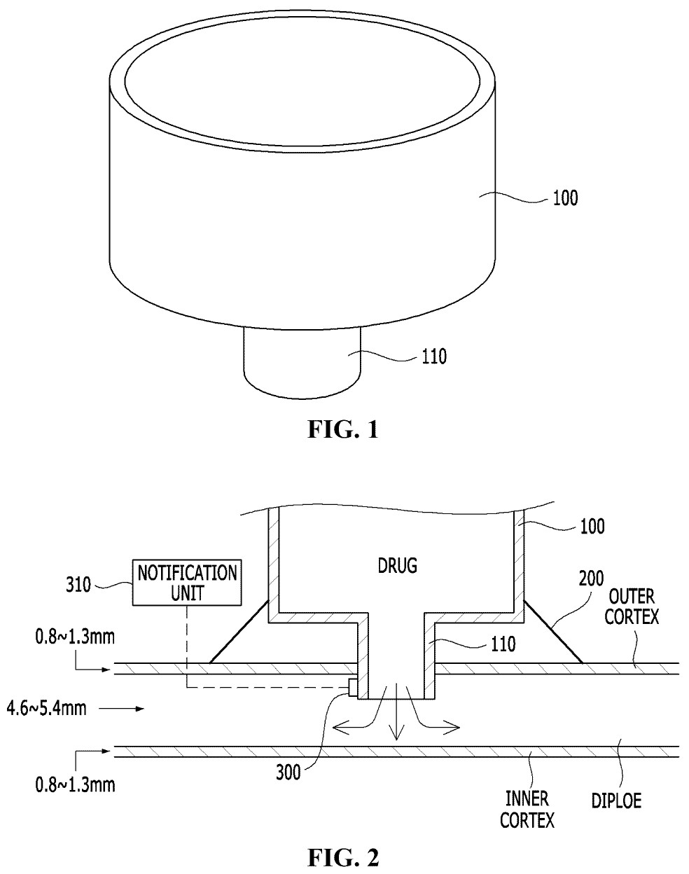

First, referring to FIGS. 1 and 2, a drug administration device according to the first embodiment of the present disclosure will be described.

As illustrated in FIGS. 1 and 2, the drug administration device according to the first embodiment of the present disclosure includes a receiving portion 100, an injection portion 110, a fixture 200, and a pressure sensor 300.

The receiving portion 100 is configured in a cylindrical shape with a space provided inside. A drug to be administered to the diploe is accommodated inside the receiving portion 100.

The shape of the receiving portion 100 is not limited to a cylindrical shape, and may have any shape as long as a space for accommodating the drug is provided inside.

The injection portion 110 is formed by extending a predetermined length to the lower portion of the receiving portion 100. Since the injection portion 110 is configured in a tube shape, the drug accommodated inside the receiving portion 100 may be discharged through the injection portion 110.

When the drug administration device is installed in the skull, the injection portion 110 should be located in a diploe between an outer cortex and an inner cortex of the skull. Therefore, the length of the injection portion 110 extending from the receiving portion 100 is formed to a length that allows the injection portion 110 to be located in the diploe.

As illustrated in FIG. 2, for example, an average thickness of a human skull is about 7 mm, the thickness of each of the outer cortex and inner cortex is 0.8 mm to 1.3 mm, and the thickness of the diploe is 4.6 mm to 5.4 mm.

Assuming that the outer cortex and inner cortex are the thinnest, the length of the injection portion 110 is approximately 0.8 mm to 6.2 mm, and by setting the length of the injection portion 110 in this way, the drug administration device may be installed corresponding to various thicknesses of the outer cortex and inner cortex.

The fixture 200 is used to fix the receiving portion 100 and the injection portion 110 to the skull. Epoxy resin or medical cement may be used for the fixture, but is not limited thereto. A material that is harmless to the human body and hardens after a certain period of time or when conditions are met and has a sealing property may be used.

The pressure sensor 300 is located at the end portion of the injection portion 110 and measures the pressure.

As described above, since the injection portion 110 of the drug administration device should be located within the diploe, it is possible to detect through the pressure sensor 300 whether the end portion of the injection portion 110 is accurately located within the diploe.

For example, if the injection portion 110 is inserted too far into the skull side and comes into contact with the inner cortex, the pressure will be different from when it is located within the diploe, so this can be detected.

In addition, when the pressure detected by the pressure sensor 300 is within a preset pressure range, a notification unit 310 may be provided to notify this.

As illustrated in FIG. 2, the drug administration device is installed in a thinned portion of the skull.

Specifically, when the outer cortex of the skull is perforated and the injection portion 110 is inserted into the perforated portion, in a state where the receiving portion 100 is located outside the skull, the end portion of the injection portion 110 is located in the diploe of the skull. In this case, since the pressure measured by the pressure sensor 300 is within a preset range, it can be known that the end portion of the injection portion 110 is accurately located within the diploe.

In this state, the fixture 200 is applied to the lower portion of the receiving portion 100 and the peripheral side of the injection portion 110 and hardened, thereby installing the drug administration device in the skull.

Therefore, the drug contained in the receiving portion 100 is administered to the diploe of the skull through the injection portion 110.

### 2. Second Embodiment

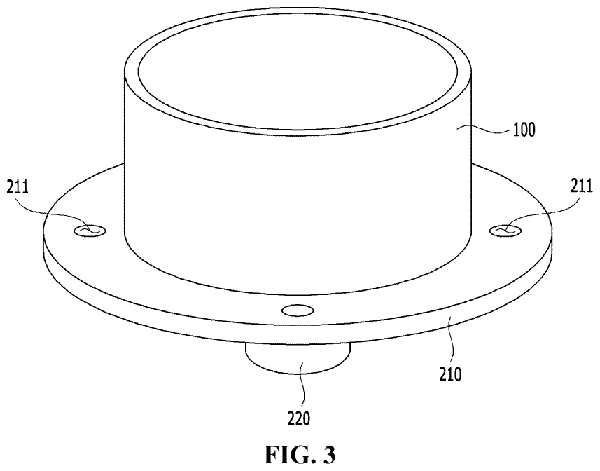

Next, referring to FIGS. 3 to 5, the drug administration device according to the second embodiment of the present disclosure will be described in detail.

In the following description, for the purpose of understanding the disclosure, differences from the first embodiment will be described, and the same drawing reference numerals will be used for the same components, and the description thereof may be omitted or simplified.

As illustrated in FIGS. 3 to 5, a drug administration device according to the second embodiment of the present disclosure includes a receiving portion 100, an injection portion 110, a holder 210, a pressure sensor 300, and a notification unit 310.

The configuration of the receiving portion 100, the injection portion 110, the pressure sensor 300, and the notification unit 310 is the same as that of the first embodiment, but in the present embodiment, the holder 210 is included instead of the fixture 200 of the first embodiment.

The holder 210 is fixed to the outside of the skull, and the receiving portion 100 is fixed by being coupled to the fixed holder 210.

The holder 210 is formed in a plate shape, and in the embodiment, it is formed in a circular plate shape, but is not limited thereto.

The insertion portion 220 is formed by extending from the lower portion of the holder 210 to a predetermined length. The insertion portion 220 has a tubular shape corresponding to the injection portion 110 of the receiving portion 100, and since the injection portion 110 of the receiving portion 100 is inserted and fixed inside the insertion portion 220, the diameter of the insertion portion 220 is formed larger than the diameter of the receiving portion 100.

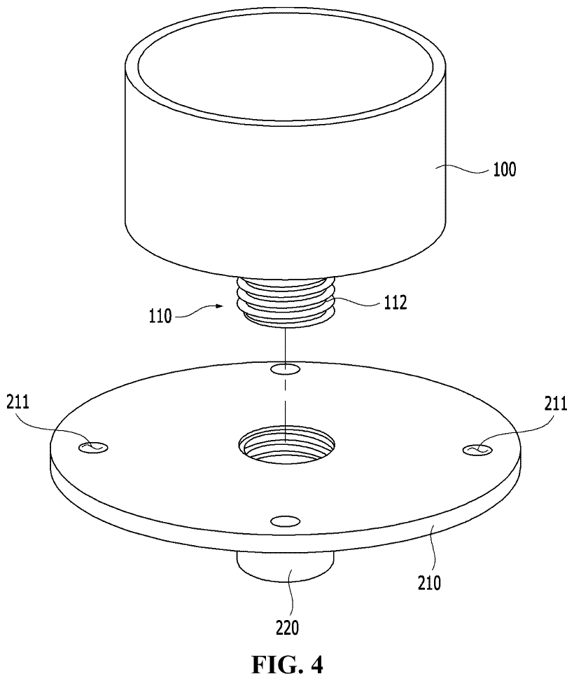

As illustrated in FIG. 4, screw threads 112 and 212 are formed on the inner surface of the insertion portion 220 and the outer surface of the injection portion 110 of the receiving portion 100, respectively, so that the injection portion 110 may be inserted while rotating into the insertion portion 220 and fixed by screw coupling.

A coupling hole 211 penetrating the holder 210 is formed on the inner side of the circumference of the holder 210. In a state where the holder 210 is positioned outside the skull, a coupling member 231 is coupled to the skull through the coupling hole 211, thereby fixing the holder 210 to the outside of the skull.

The coupling member 231 may be a screw, but is not limited thereto.

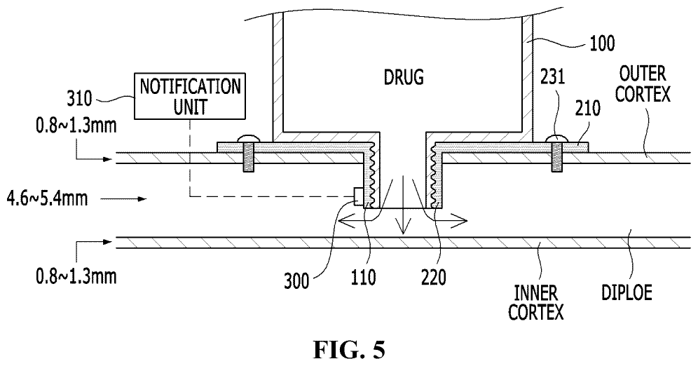

As illustrated in FIG. 5, the drug administration device according to the present embodiment is installed in the part where the skull is thinned.

Specifically, after perforating the outer cortex of the skull, the insertion portion 220 of the holder 210 is inserted into the perforated portion. Moreover, in a state where the holder 210 is positioned outside the skull, the end portion of the insertion portion 220 is positioned in the diploe within the skull, and the holder 210 is coupled to the outer surface of the skull, i.e., the outer cortex, using the coupling member 231.

Then, by inserting the injection portion 110 of the receiving portion 100 into the insertion portion 220 of the holder 210, the end portion of the injection portion 110 is positioned inside the diploe, and the receiving portion 100 is positioned outside the skull in a state of being fixed to the holder 210.

Thereby, the drug accommodated in the receiving portion 100 is administered to the diploe within the skull through the injection portion 110.

### 3. Third Embodiment

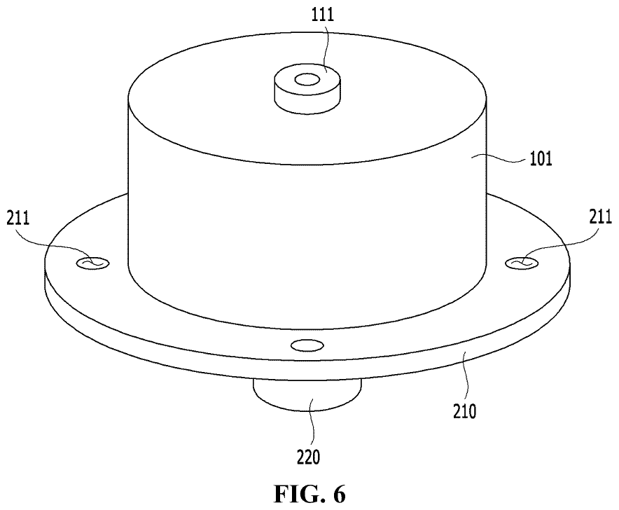

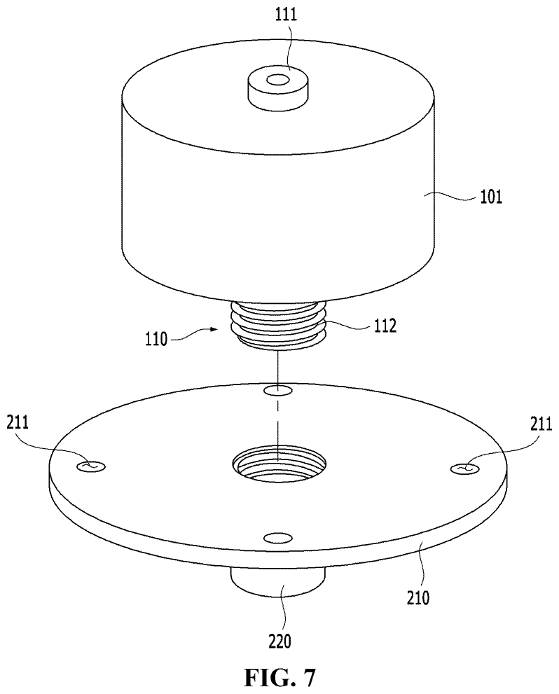

Next, with reference to FIGS. 6 to 8, a drug administration device according to a third embodiment of the present disclosure will be described in detail.

Likewise, in the following description, for the purpose of understanding the disclosure, the differences from the first and second embodiments will be described, and the same drawing reference numerals will be used for the same components, and the descriptions thereof may be omitted or simplified.

As illustrated in FIGS. 6 to 8, the drug administration device according to the third embodiment of the present disclosure includes a main body 101, an injection tube 111, a holder 210, a pressure sensor 300, and a notification unit 310.

The configurations of the holder 210, the pressure sensor 300, and the notification unit 310 are the same as those of the second embodiment, but in the present embodiment, the main body 101 is included instead of the receiving portion 100 of the first and second embodiments.

The main body 101 has the same shape and form as the receiving portion 100, but since there is no space for receiving the drug inside, the drug is not received, and the injection tube 111 is located.

The injection portion 110 is formed by protruding and extending a predetermined length from the lower portion of the main body 101.

The injection tube 111 is positioned to penetrate the main body 101 and the injection portion 110. Specifically, the injection tube 111 is positioned to penetrate from the upper portion of the main body 101 to the end portion of the injection portion 110.

In the present embodiment, the drug contained in the receiving portion 100 is not discharged and administered, but is injected from the outside into the injection tube 111 to be administered.

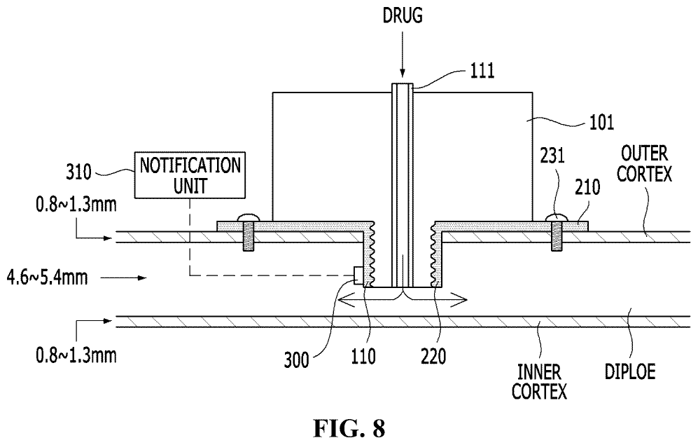

As illustrated in FIG. 8, the drug administration device according to the present embodiment is installed in a part where the skull is thinned.

As in the second embodiment, after perforating the outer cortex of the skull, the insertion portion 220 of the holder 210 is inserted into the perforated portion. Moreover, in a state where the holder 210 is positioned outside the skull, the end portion of the insertion portion 220 is positioned in the diploe within the skull, and the holder 210 is coupled to the outer surface of the skull, i.e., the outer cortex, using the coupling member 231.

Then, by inserting the injection portion 110 of the main body 101 into the insertion portion 220 of the holder 210, the end portions of the injection portion 110 and the injection tube 111 are positioned inside the diploe, and the main body 101 is positioned outside the skull in a state of being fixed to the holder 210.

Then, the drug injected from the upper portion of the injection tube 111 is administered to the diploe within the skull through the injection tube 111.

### 4. Effect of Present Disclosure

Hereinafter, the effects of the present disclosure will be explained by comparing intracalvariosseous (hereinafter referred to as ICO) administration and intravenous (hereinafter referred to as IV) administration according to the present disclosure.

First, nine types of drugs to be administered were prepared.

Chlorpromazine hydrochloride (hereinafter, CPZ), risperidone (hereinafter, RIS), temozolomide (hereinafter, TMZ), paclitaxel (PTX), donepezil (hereinafter, DPZ), rivastigmine i-tartrates (hereinafter, RVG), risperidone-d4, gamma-aminobutyric acid-d6 (hereinafter, GABA), glutathione (glycine-13C2, 15 N) sodium salt (hereinafter, GSH), and [UL-13C12] sucrose (UL-13C12, UL-13C6], glucose (13C), and fructose (D-[UL-13C6]) (SUC, SUC) were prepared.

The experiment was performed with 5-week-old male rats weighing 20 to 25 g. To ensure statistical independence, only male rats were used in each experiment.

The animal experiments on the above 9 drugs were conducted as follows: IV (intravenous) administration and ICO (intracalvariosseous) administration groups by compound: (1) CPZ-IV and ICO, (2) RIS-IV and ICO, (3) DPZ-IV and ICO, (4) RVG-IV and ICO, (5) TMZ-IV and ICO, (6) PTX-IV and ICO, (7) GABA-IV and ICO, (8) GSH-IV and ICO, (9) SUC-IV and ICO. Eight rats were randomly assigned to the IV and ICO administration groups for each compound and were administered continuously from the next day (n=4).

Moreover, as the drug administration device according to the present disclosure, the drug administration device of the first embodiment to be installed on the skull of the ICO administration group rats was manufactured and tested, but of course, the drug administration devices according to the second and third embodiments can also be applied to the experiment.

The drug administration device (total height 5.5 mm) in which the receiving portion 100 and the injection portion 110 are formed integrally to fit the rat skull was manufactured using a 3D printer.

The 3D printing material was USP Class VI certified VisiJet M3 Crystal (3D Systems, USA), and the injection portion 110 was made to have an outer diameter of 1.3 mm, an inner diameter of 0.8 mm, and a height of 0.5 mm in consideration of the size and thickness of the rat skull.

In order to perform the experiment, the drug administration device of the present disclosure should be installed on the rats in the ICO administration group, and a guide cannula for a microdialysis probe for measuring the concentration of drugs in the brain should be installed on the rats in both the ICO administration group and the IV administration group.

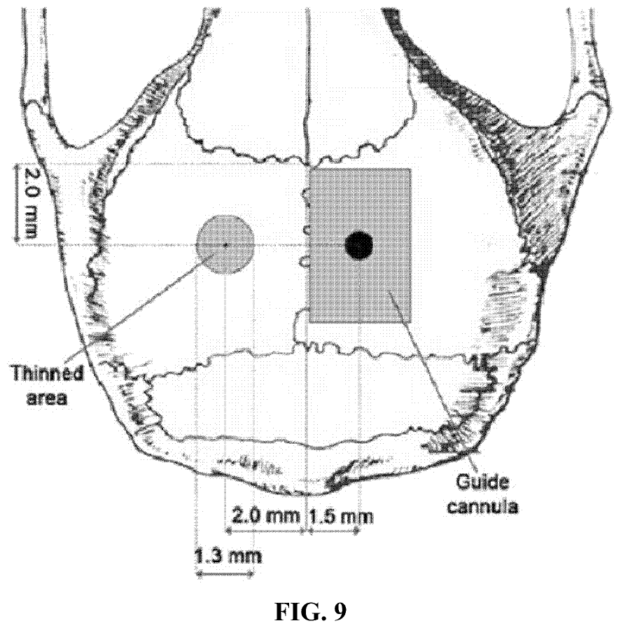

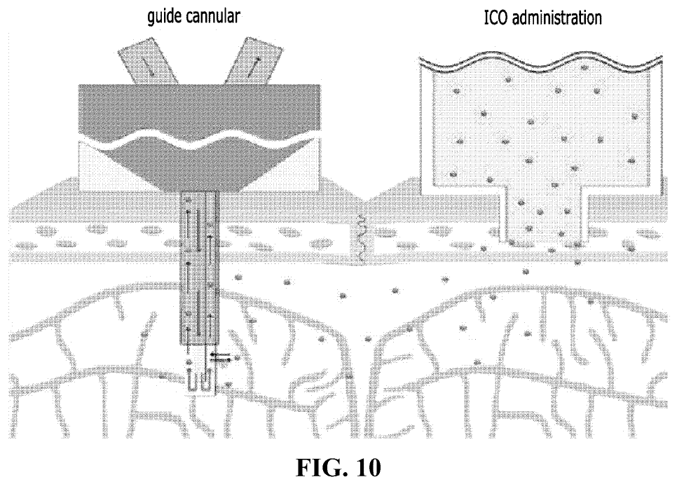

As illustrated in FIG. 9 and FIG. 10, the drug administration device was installed on the left side of the skull of the rats in the ICO administration group, and the guide cannula was installed on the right side, while only the guide cannula was implanted in the skull of the IV administration group.

In order to install the drug administration device in the skull of the rats, the rats were anesthetized with isoflurane and then skull thinning was performed.

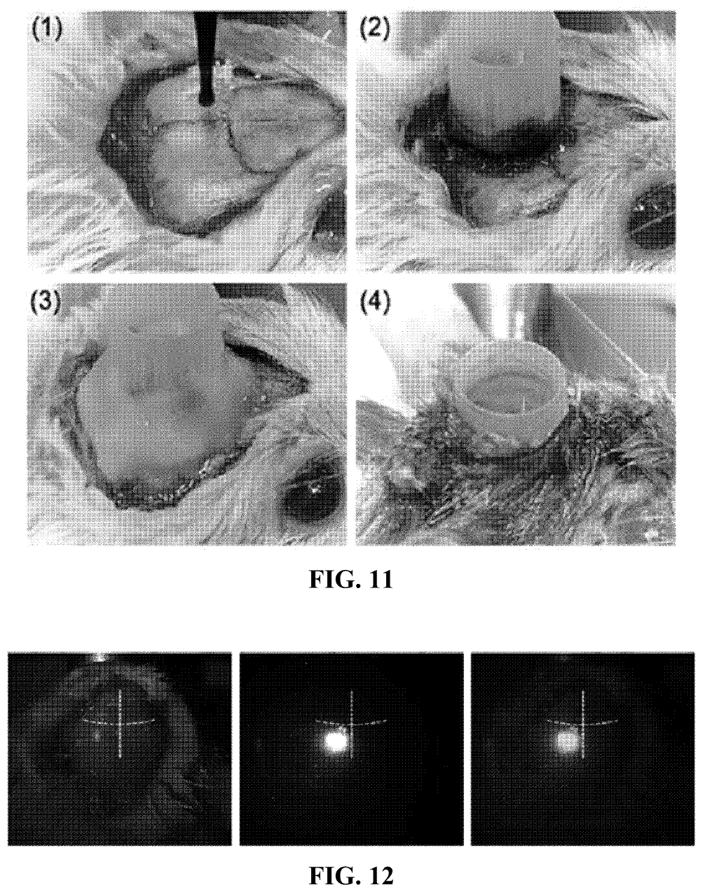

As illustrated in FIG. 11, the skull of the rats was thinned, that is, a hole of approximately 1.35 mm was drilled in the outer cortex using a drill robot and a control system.

After thinning the skull, bone dust was removed, and then the injection portion 110 was inserted into the thinned portion to position the drug administration device, sealed with epoxy adhesive and dental cement, and the skin wound was sutured with suture.

As a result, the injection portion 110 of the drug administration device was inserted into the diploe to a depth of 0.2 mm.

Next, the integrity of the drug administration device installed in the skull of the rat was tested by near-infrared (NIR) fluorescence imaging using indocyanine green (IGC).

The diffusion of ICG into the rat skull was observed by the near-infrared fluorescence imaging of the drug administration device installed in the thinned rat skull.

50 μl of ICG (500 μg/ml in saline) was administered to the rat with the drug administration device installed. After 24 hours, the rat was anesthetized, placed in a stereotaxic position, and the scalp was cut. For the ICG-NIR fluorescence imaging, a 780 nm laser with 150 mW output was set at a distance of 80 mm and an angle of 20° from the target through a C-clamp on a Z stand for a laser spot size of 15 mm width and 25 mm length, and fluorescence images were recorded at 1 frame per second using basedcam2 software while NIR light was administered through the 780 nm laser.

As illustrated in FIG. 12, when observing the fluorescence image, the ICG signal was brightly lit in the left skull where the drug administration device was installed, whereas no signal was observed in the right skull.

In addition, no leakage was observed in the area sealed with the fixture 200, which clearly indicates that the ICG diffused into the double space of the left skull where the drug administration device was installed.

Then, in order to implant the microdialysis guide cannula, the rats in the ICO and IV administration groups were anesthetized with isoflurane, and a small hole was drilled in the skull, and the guide cannula was inserted into the right hippocampus (see FIGS. 9 and 10). The inserted guide cannula was fixed with dental cement, and the skin wound was sutured, and then the rats were allowed to recover from the surgery in the cage for one day.

Before the drug administration, the membrane of the microdialysis probe was soaked in ethanol and artificial cerebrospinal fluid (aCSF) buffer was perfused through the microdialysis probe at a flow rate of 0.5 μl/min for 1 hour for stabilization.

Nine types of drugs (CPZ, RIS, DPZ, RVG, TMZ, PTX, GABA, GSH, and SUC) were intravenously injected into each IV administration group through the tail vein at a dose of 0.5 mg/kg.

In addition, the ICO administration group was administered at a dose of 5 mg/kg by filling the drug into the receiving portion 100 of the drug administration device.

The perfusate of the microdialysis probe was collected every 30 minutes for 4 hours between 8 and 24 hours after administration.

The dialysate samples were stored at 80° C. until the time of LC-MS/MS analysis (tandem mass spectrometry). Blood samples (30 μl) were administered to 0.6 ml heparinized tubes and collected from the saphenous vein at 2, 5, 10, 30, 60, 120, 240, 480, and 1440 minutes.

Blood samples were immediately centrifuged at 10,000 rpm for 10 minutes at 4° C. to recover plasma. In the case of TMZ, blood samples collected after administration were immediately centrifuged and then 10 μl of plasma was acidified with 2 μl of 0.1% formic acid.

Brains were excised 24 hours after administration, and PBS (phosphate buffer saline) corresponding to the weight of each tissue sample was added, followed by homogenization using a homogenizer.

Plasma and homogenized brain tissue samples were stored at 80° C. until LC-MS/MS analysis.

to compare the results according to the route of administration of nine types of drugs (CPZ, RIS, SUC, TMZ, PTX, DPZ, RVG, GABA, and GSH), LC-MS/MS analysis and plots over time were performed. The areas under the curve in plasma (AUCₚₗₐₛₘₐ) and interstitial fluid (ISF) concentration-time curves (AUC_{ISF}) were determined by noncompartmental analysis using WinNonlin 2.1. Brain concentration (C_{br}) in the whole brain at 24 hours was corrected for the plasma volume of the corresponding organ using the Expression below, which was determined using LC-MS/MS.

$${C}_{b r} = {C}_{b r} {,}_{quant , 24 h} - ( {V}_{0} \times {C}_{plasma , 24 h} ) ,$$

In the above Expression, C_{br, quant, 24 h} is the brain drug concentration (ng/g) at 24 hours quantified by LC-MS/MS, V₀ is the plasma volume of the corresponding organ (μl/g), C_{plasma, 24 h} at 24 hours is the plasma concentration (ng/ml), and the V₀ value for the brain was selected as 9.3±1.1 μl/g.

Compared to IV administration, the extent of brain uptake after ICO administration was expressed as brain availability (F_{br}), which is similar to systemic bioavailability (F), and was calculated as the ratio of AUC_{ISF}, IV and AUC_{ISF, ICO} using the Expression below.

$${F}_{br} = {AUC}_{ISF , ICO} / {AUC}_{ISF , IV ,}$$

In the above Expression, AUC_{ISF,ICO} and AUC_{ISF,IV} are the areas under the curves in the brain interstitial fluid-time plot (AUC_{ISF}) up to 24 hours after ICO administration and IV administration, respectively.

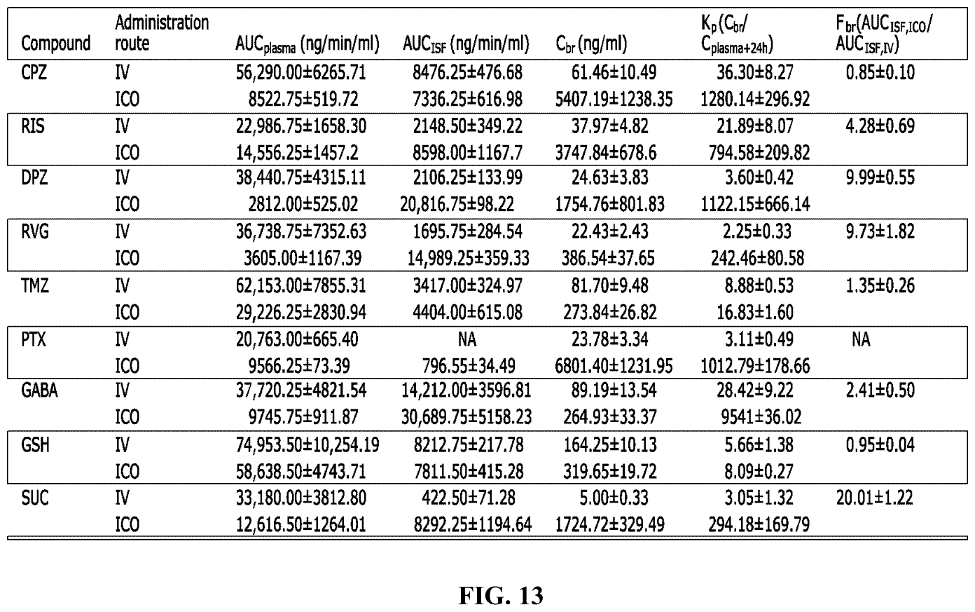

The pharmacokinetic parameters including AUCₚₗₐₛₘₐ, AUC_{ISF}, brain drug concentration (C_{br}), brain/plasma ratio (Kₚ) at 24 hours and in vivo brain availability (F_{br}) of nine compounds after IV and ICO administration, corrected as described above, are summarized in FIG. 13 (mean±SEM, n=4).

All data were expressed as mean±SEM (standard error of measurement), and each data represented the mean of four individual experiments for each group. The two samples were analyzed for significant differences using student's t-test, and statistical significance was indicated as *p<0.05 and **p<0.01 in FIG. 13.

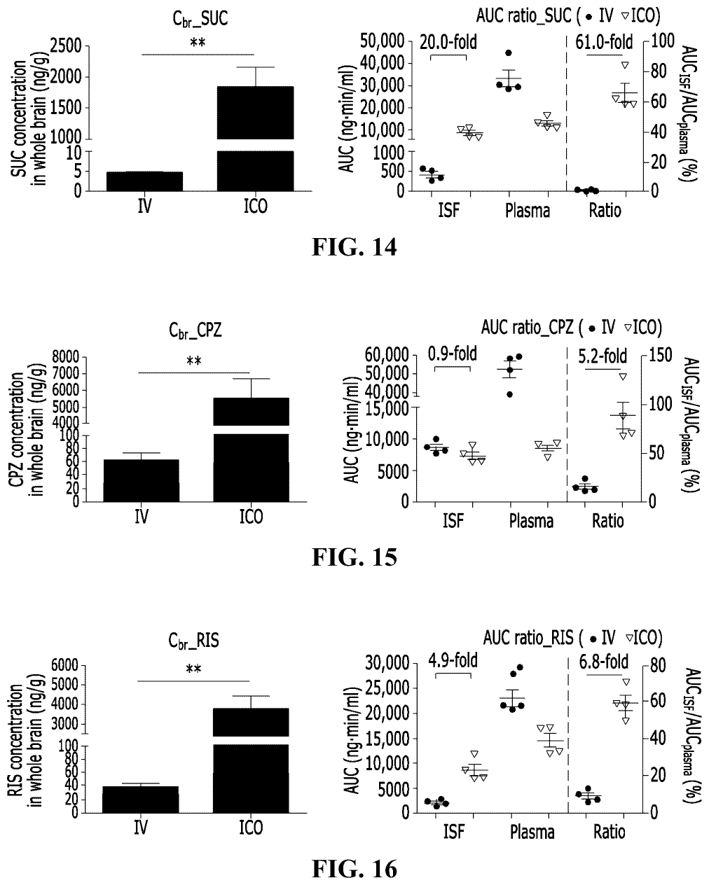

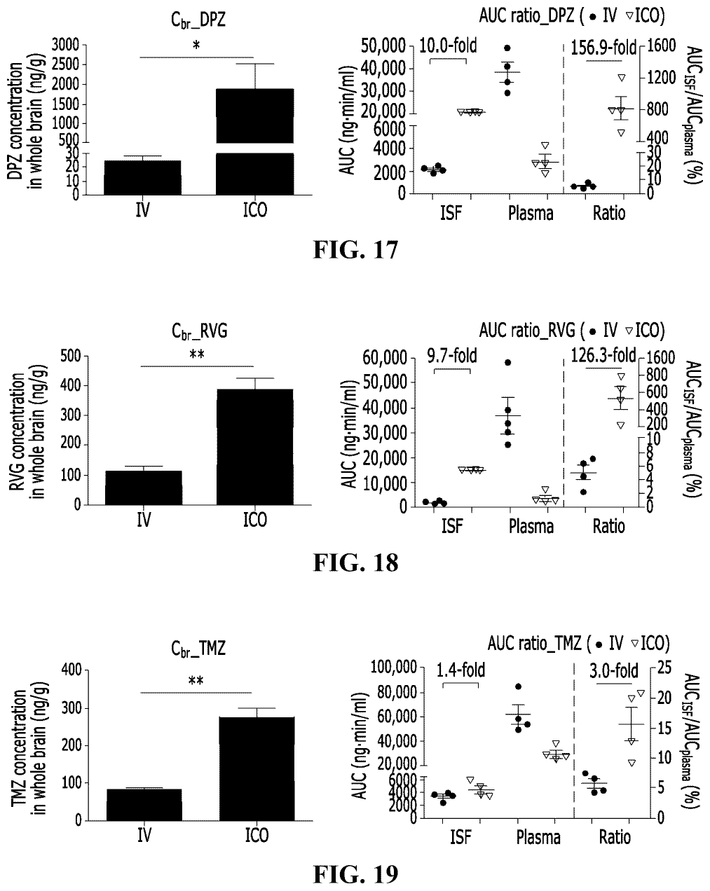

FIGS. 14 to 22 illustrate the total brain drug concentration (C_{br}), AUC value, and the ratio of AUC_{ISF} to AUCₚₗₐₛₘₐ at 24 hours after ICO and IV administration of nine types of drugs (SUC, CPZ, RIS, DPZ, RVG, TMZ, PTX, GABA, and GSH), respectively.

Referring to FIGS. 14 to 22, the statistical analysis results for seven types of compounds (SUC, RIS, DPZ, RVG, TMZ, PTX, and GABA) showed that the AUCₚₗₐₛₘₐ of the ICO administration group was relatively lower than that of the IV administration group, whereas the AUC_{ISF} of the ICO administration group tended to be relatively higher than that of the IV administration group.

CPZ and GSH showed that the AUCₚₗₐₛₘₐ of the ICO administration group was relatively lower than that of the IV administration group, whereas the AUC_{ISF} of the ICO administration group was no different from that of the IV administration group.

The AUC ratio (%, AUC_{ISF}/AUCₚₗₐₛₘₐ) was significantly higher in the ICO administration group than in the IV administration group, ranging from 3.0 times (TMZ) to 156.9 times (DPZ), except for PTX and GSH.

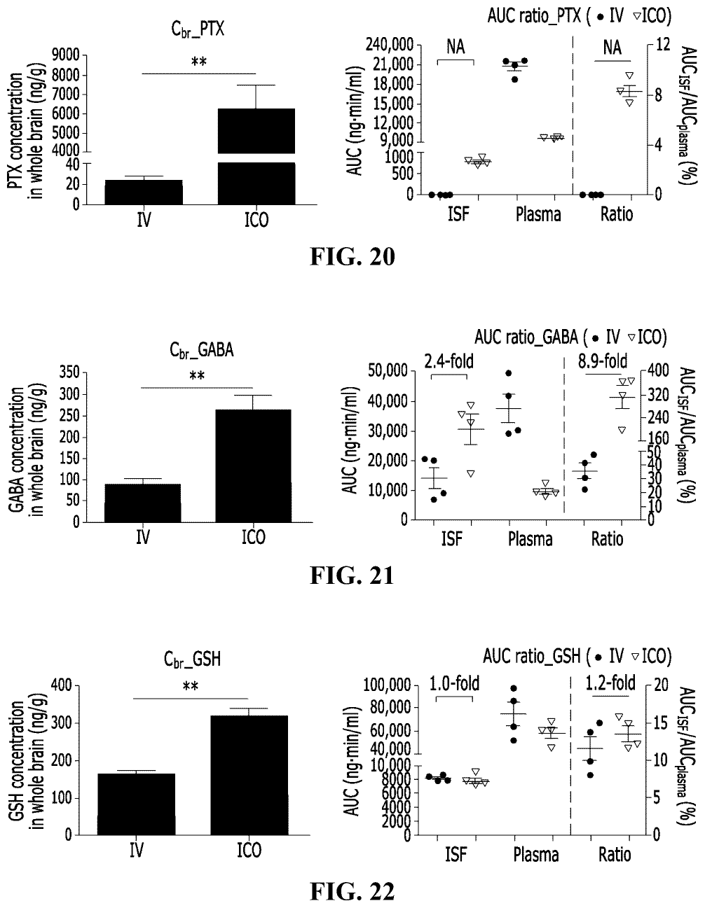

The brain availability (F_{br}), similar to the systemic bioavailability (F), was illustrated in the ratio of AUC_{ISF,IV} and AUC_{ISF,ICO} in FIG. 13. In particular, in the case of PTX, the ISF concentration was below the limit of detection (LOD) at all time points after IV administration and was not analyzed (NA), whereas it was detected after ICO administration and calculated as 796.55 ng/min/ml of AUC_{ISF} (see FIG. 20). It can be seen that PTX, which could not enter the brain sufficiently with IV administration, was able to do so with ICO administration.

In addition, C_{br} which was a parameter representing the actual brain drug concentration at 24 hours after all drug administration, was significantly higher in the ICO administration group than in the IV administration group by 85.1 times (CPZ), 115.2 times (RIS), 64.3 times (DPZ), 18.6 times (RVG), 3.4 times (TMZ), 294.1 times (PTX), 3.1 times (GABA), 3.4 times (GSH), and 342.0 times (SUC).

These results demonstrate that ICO administration may provide a special environment for drugs to enter the brain.

In addition, the brain-to-plasma ratio (Kₚ) at 24 hours after all drug administration was significantly higher in the ICO administration group than in the IV administration group by 35.3 times (CPZ), 36.3 times (RIS), 311.3 times (DPZ), 107.8 times (RVG), 1.9 times (TMZ), 325.7 times (PTX), 3.4 times (GABA), 1.4 times (GSH), and 96.5 times (SUC).

This suggests that brain accumulation may be processed differently after ICO administration compared to IV administration, which relies solely on the blood-brain barrier (BBB) delivery route for brain drug delivery.

Additionally, in vitro brain penetration of selected drugs was evaluated to investigate the relationship between in vivo brain uptake and molecular properties of the drugs.

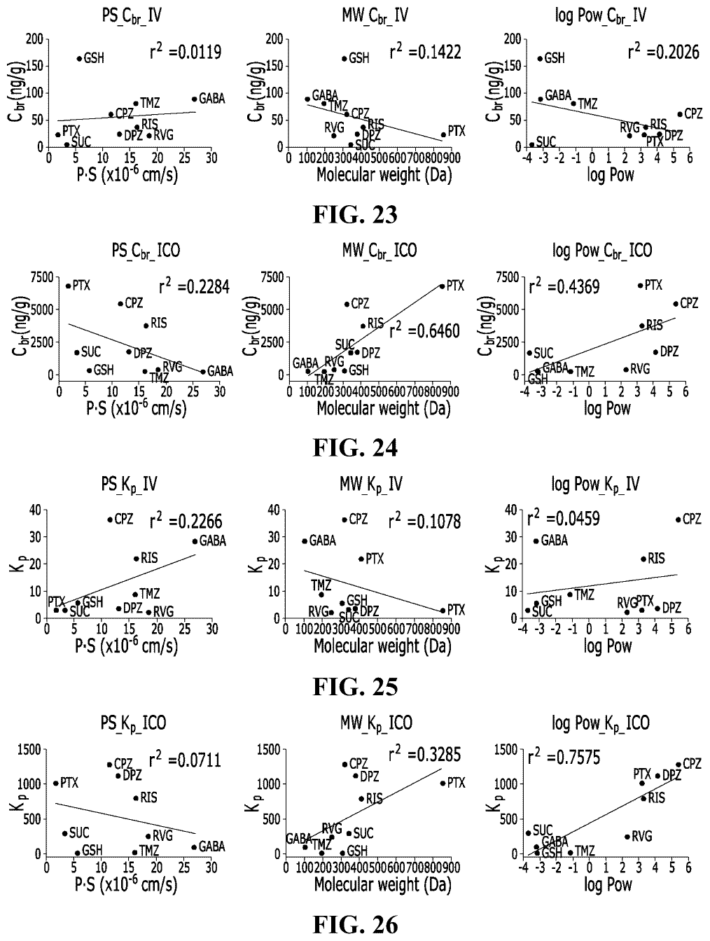

FIGS. 23 and 24 illustrate relationships between experimental brain drug concentrations (C_{br}) and in vitro experimental BBB permeability product-surface (PS) values, molecular weight (MW), and octanol-water partition coefficient (log Pow) at 24 hours after IV and ICO administration.

Moreover, FIGS. 25 and 26 illustrate the relationship between the brain/plasma concentration ratio (Kₚ) and the experimental BBB permeability product-surface (PS) value, molecular weight (MW), and octanol-water partition coefficient (log Pow) calculated at 24 hours after IV and ICO administration.

In the IV administration group, higher PS values resulted in higher C_{br}, whereas higher MW and log pow values resulted in lower C_{br} (see FIG. 24).

Conversely, in the ICO administration group, the lower the PS value, the higher the C_{br}, and the lower the MW and log pow, the lower the C_{br} (see FIG. 25).

Although no clear trend was observed from the r² value (0.0119-0.6460), the regression lines between the IV administration group and the ICO administration group clearly illustrated opposite slopes.

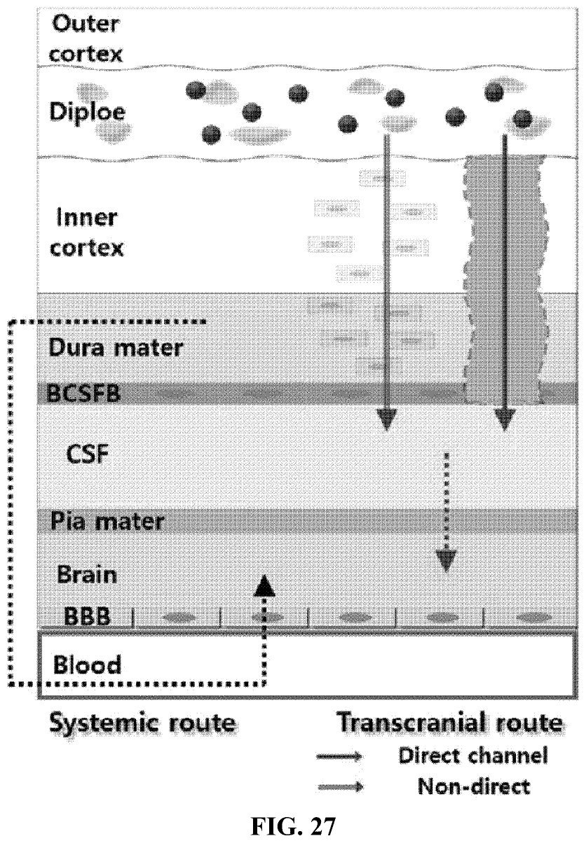

Similarly, the Kₚ regression lines for the IV and ICO administration groups showed clearly opposite slopes in the relationship between PS and MW (see FIGS. 26 and 27).

For the relationship between Kₚ and log Pow (see FIGS. 26 and 27), the slope directions of the regression lines for IV and ICO administration were the same (r²-IV: 0.0587, r²-ICO: 0.7381), but these results indicated that the brain uptake of the drug after ICO administration was processed in a different way, resulting in higher brain exposure and drug accumulation compared to IV administration.

As above, the regression lines of the correlation analysis of total brain drug concentration (C_{br}) and brain/plasma ratio (Kₚ) at 24 hours related to molecular characteristics showed opposite slope directions for ICO and IV administration.

For IV administration, which involves direct injection into the bloodstream, the drug should cross the BBB to enter the brain, so brain concentrations are affected by blood concentrations of the drug.

It is reported that hydrophilic compounds have limited passive transport of drug molecules across the BBB, whereas lipophilic drugs with molecular weights less than 400 to 600 Da can enter the brain relatively easily.

This is consistent with the results of the IV administration correlation data, where lower PS values correspond to lower C_{br}, and lower MW and log Pow correspond to higher C_{br}.

However, in the case of ICO administration to the skull diploe, which showed contradictory results compared to IV administration, it is conceivable that brain uptake of the compound after administration is processed through a different route than the systemic route, which involves only the blood-brain route.

FIG. 27 illustrates the major transcranial routes of molecules penetrating from the skull cortex into the brain following ICO administration.

As illustrated in FIG. 27, molecules in the diploe may be delivered to the brain via the transcranial route, including direct channels and non-direct channels, as well as the systemic route.

The meninges are made up of three layers, including the dura mater, arachnoid mater, and pia mater, which surround the brain and spinal cord.

The arachnoid barrier cell layers are distinguished by continuous and complex tight junctions that form the epithelial blood-cerebrospinal fluid barrier (BCSFB) between the dura mater and the subarachnoid space.

Cerebrospinal fluid (CSF) fills the subarachnoid space below the arachnoid barrier, and the pia mater is a thin, transparent layer that separates the cerebrospinal fluid from the brain.

Because the pia mater has no rigid junctions, molecules can easily enter the brain when the molecules reach the cerebrospinal fluid, and the cerebrospinal fluid accesses the skull bone marrow through direct channels.

The direct channel allows the bone marrow direct access to the cerebrospinal fluid, allowing a route for drug delivery from the skull to the brain.

Although the meninges, which provide the cerebrospinal fluid barrier, may be considered an obstacle to drug delivery from the skull to the brain by the ICO administration, the above experimental results clearly illustrate that the skull is not completely impermeable to drug delivery to the brain when administering drugs through the diploe using the drug administration device of the present disclosure.

In addition, the intracranial drug administration device using the drug administration device according to the present disclosure and the effect of intracalvariosseous drug administration using the same can be applied to drugs for treating central nervous system disorders and other BBB-impermeable drugs.

The drug administration device according to the present disclosure provides an innovative and advantageous route of the drug for the effective treatment of brain diseases by allowing the drug to be administered into the skull, thereby solving both the problems of invasive procedures and complications of existing intracerebroventricular or intrathecal drug administration and the low drug delivery rate of intravenous administration.

Although the preferred embodiments of the present disclosure have been described above, the present disclosure is not limited to the specific embodiments described above. That is, those skilled in the art to which the present disclosure pertains may make numerous changes and modifications to the present disclosure without departing from the spirit and scope of the appended claims, and equivalents of all such appropriate changes and modifications should also be considered to fall within the scope of the present disclosure.

1. A device for administering a drug to a diploe within a skull, the device comprising:
a receiving portion (100) having a space provided therein for receiving the drug; and
an injection portion (110) formed by extending from the receiving portion (100) to a predetermined length and allowing the drug received in the receiving portion (100) to be discharged,
wherein an end portion of the injection portion 110 is located in the diploe within the skull in a state where the receiving portion (100) is located outside the skull.

2. The device according to claim 1, further including a fixture (200) located on a peripheral side of the receiving portion (100).

3. A device for administering a drug to a diploe within a skull, the device comprising:
a receiving portion (100) having a space provided therein for receiving the drug;
an injection portion (110) formed by extending from the receiving portion (100) to a predetermined length and allowing the drug received in the receiving portion (100) to be discharged;
a holder (210) fixed to an outside of the skull;
an insertion portion (220) formed by extending from the holder (210) to a predetermined length and having a diameter larger than that of the injection portion (110); and
a coupling hole (211) located on a peripheral side of the holder (210),
wherein the holder (210) is fixed to the outside of the skull by a coupling member (231) coupled to the coupling hole (211), and in a state where the insertion portion (220) is located in the diploe within the skull, the injection portion (110) is inserted into the insertion portion (220) so that the receiving portion (100) is fixed to the outside of the skull, and an end portion of the injection portion (110) is located in the diploe within the skull.

4. A device for administering a drug to a diploe within a skull, the device comprising:
a main body (101);
an injection portion (110) extending from the main body (101) to a predetermined length;
an injection tube (111) installed by penetrating an end portion of the injection portion (110) from an upper portion of the main body (101);
a holder (210) fixed to an outside of the skull;
an insertion portion (220) extending from the holder (210) to a predetermined length and formed with a diameter larger than a diameter of the injection portion (110); and
a coupling hole (211) located on a peripheral side of the holder (210),
wherein the holder (210) is fixed to the outside of the skull by a coupling member (231) coupled to the coupling hole (211), and in a state where the insertion portion (220) is located in the diploe within the skull, the receiving portion (100) is fixed to the outside of the skull, and the injection portion (110) is inserted into the insertion portion (220) so that an end portion of the injection tube (111) is located in the diploe within the skull.

5. The device according to claim 3, further comprising the coupling hole (211) located on the peripheral side of the holder (210),
wherein the holder (210) is fixed to the outside of the skull as a screw penetrates the coupling hole (211) and is coupled to the skull.

6. The device according to claim 3, further comprising screw threads (112 and 212) formed on an outer surface of the injection portion (110) and an inner surface of the insertion portion (220), respectively,
wherein the injection portion (110) and the insertion portion (220) are rotatably coupled by the screw threads (112 and 212).

7. The device according to claim 1, wherein the predetermined length of the injection portion (110) is 0.8 to 6.2 mm.

8. The device according to claim 3, wherein the predetermined length of the injection portion (110) is 0.8 to 6.2 mm added to a thickness of the holder (210).

9. The device according to claim 1, further comprising:
a pressure sensor (300) located on a side of an end portion of the injection portion (110); and
a notification unit (310) that notifies when a pressure measured by the pressure sensor (300) is within a preset pressure range.

10. The device according to claim 4, further comprising the coupling hole (211) located on the peripheral side of the holder (210),
wherein the holder (210) is fixed to the outside of the skull as a screw penetrates the coupling hole (211) and is coupled to the skull.

11. The device according to claim 4, further comprising screw threads (112 and 212) formed on an outer surface of the injection portion (110) and an inner surface of the insertion portion (220), respectively,
wherein the injection portion (110) and the insertion portion (220) are rotatably coupled by the screw threads (112 and 212).

12. The device according to claim 4, wherein the predetermined length of the injection portion (110) is 0.8 to 6.2 mm added to a thickness of the holder (210).

13. The device according to claim 3, further comprising:
a pressure sensor (300) located on a side of an end portion of the injection portion (110); and
a notification unit (310) that notifies when a pressure measured by the pressure sensor (300) is within a preset pressure range.

14. The device according to claim 4, further comprising:
a pressure sensor (300) located on a side of an end portion of the injection portion (110); and
a notification unit (310) that notifies when a pressure measured by the pressure sensor (300) is within a preset pressure range.
